(19) United States
(12) Patent Application Publication
(10) Pub. No.: US 2021/0228074 A1
(43) Pub. Date: Jul. 29, 2021

(54) PERIMETRY TESTING USING MULTIMEDIA

(71) Applicant: NOVA SOUTHEASTERN UNIVERSITY, Fort Lauderdale, FL (US)

(72) Inventor: Ava K. Bittner, Fort Lauderdale, FL (US)

(73) Assignee: NOVA SOUTHEASTERN UNIVERSITY, Fort Lauderdale, FL (US)

(21) Appl. No.: 16/080,472

(22) PCT Filed: Feb. 28, 2017
(86) PCT No.: PCT/US2017/019896

§ 371 Date: Aug. 28, 2018

Related U.S. Application Data
(60) Provisional application No. 62/301,776, filed on Mar. 1, 2016.

(51) Int. Cl. A61B 3/00; A61B 3/024; G16H 50/20

(52) U.S. Cl. CPC A61B 3/0091; G16H 50/20; A61B 3/0041; A61B 3/024

(57) ABSTRACT

A device for perimetry visual field testing of a patient includes a perimeter having a target fixation location; at least one video player positioned to display a video clip at the target fixation location; an audio clip player positioned to play one or more audio clips to be heard by the patient during visual field testing; and an electronic controller associated with the video display to cause a particular video clip to play during testing. The related method of visual field testing using a perimetry VF tester includes: positioning a video display at a fixation location within a perimeter of the tester; playing at least one video clips upon the video display during testing; and playing one or more audio clips corresponding to at least one of the video clip being displayed and a current stage of testing.

### FIELD OF THE DISCLOSURE

The disclosure relates to a system and method for visual field testing of patients, and in particular, perimetry testing using a video display to present target fixation objects.

### BACKGROUND

Visual field testing is conducted to detect dysfunction in central and peripheral vision due to, for example, glaucoma, stroke, brain tumor, or a neurological problem.

In perimetry visual field testing, the patient fixates or maintains focus on a fixed object directly ahead. While the patient is doing so, a stimulus such as an object or a light is presented elsewhere in the field of vision, and the patient indicates when it is visible to the patient, if at all, at each location in which it is presented. The stimulus is presented at locations that are unpredictable to the patient, until all locations of interest have been tested.

Static perimetry tests the sensitivity of different locations in the field of view by presenting lights of varying brightness in a single location at a time and the patient reports each time the light is seen. In Kinetic perimetry, commonly carried out using a Goldmann or Octopus perimetry device, a stimulus such as a test light is moved by an examiner (perimetrist) or the instrument program from outside the peripheral vision of the patient towards the center of vision or from non-seeing to seeing areas of vision until it is detected by the patient. The test may be repeated using test stimuli of different brightness levels or lights of a different size.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is a front view of a Humphrey visual field testing apparatus, configured in accordance with the disclosure with a video display for target fixation;

FIG. 2 is a closer, front view, of the display of FIG. 1, illustrating an example cartoon and video controls;

FIG. 3 is a diagram of a side view of a perimeter of a VF tester, including a front projection video display of the disclosure;

FIG. 4 is a diagram of a side view of a perimeter of a VF tester, including two front projectors of the disclosure;

FIG. 5 is a diagram of a side view of a perimeter of a VF tester, including two rear projectors of the disclosure;

FIG. 6 is a diagram of a side view of a perimeter of a VF tester, including a flexible video display inserted into an interior of the perimeter;

FIG. 7 is a diagram of a side view of a perimeter of a VF tester, including a flexible video display applied to an exterior of a light transmissive perimeter;

FIG. 8 is a diagram of a side view of a perimeter of a VF tester that is formed from a flexible video display;

FIG. 9 depicts example cartoons or images that can be displayed on one or more video displays of the disclosure;

FIG. 10 depicts a video image shown from a front of a perimeter, the video image created using any of the embodiments of FIGS. 3-8, the image including a fixation target and imagery;

FIG. 11 depicts a video image shown from a front of a perimeter, the video image created using any of the embodiments of FIGS. 3-8, the image including a fixation target that is different than that of FIG. 10, and illustrating that a stimulus object can be presented at any location, or desired locations, upon the perimeter;

FIG. 11A depicts the stimulus object of FIG. 11, at several brightness levels;

FIG. 12 depicts a mechanical VF tester including a target fixation display of the disclosure;

FIG. 13 depicts the tester of FIG. 12, configured in accordance with the disclosure with a target fixation display, computing device controller, and second, movable display;

FIG. 14 depicts the tester of FIG. 14, configured with a flexible video display of the disclosure extending along a perimeter band of the tester; and

FIG. 15 is a diagram of an electronic computing device, some or all of which can be used to carry out the disclosure.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

Poor visual field (VF) testing reliability can be a problem, since incorrect or inconsistent results can potentially lead to loss of vision due to failure to timely detect correctable problems. At least, inconsistent results can create a necessity for retesting. For this reason, VF tests are not a sole or primary means of testing children, as children typically have difficulty maintaining concentration and providing reliable feedback during testing. This leads to inconsistent results and challenges in interpreting test results.

In accordance with the disclosure, a visual field tester is provided with a multimedia interface for maintaining the attention of the individual completing VF testing. More particularly, in an embodiment, physically small video images or clips are presented at the location where the test subject is required to maintain focus during the test. In addition, one or more audio sequences or clips are played at appropriate times during testing to educate, direct, and reward the individual before, during, and after testing. The audio clips can be coordinated with the video clips so that it appears that one or more characters in the video clip are speaking.

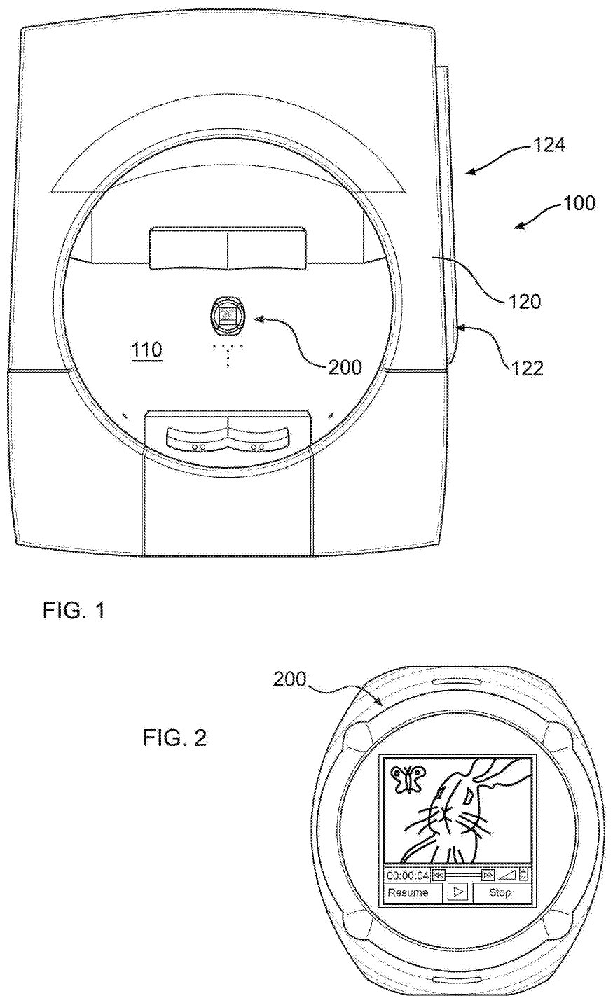

With reference to FIG. 1, in a system 100 of the disclosure, a VF tester 120 is depicted, in this example a Humphrey type tester, in which a 1.5 inch or smaller video display 200 is mounted at the target fixation location at the center of a visual field perimeter 110. Display 200 can be of any display technology, including LCD, LED, OLED, or other video display technology that is known or hereinafter developed. An electronic controller can form a part of display 200, whereby display 200 is an integral video player unit, or can be provided by a separate computing device (700), which is connected to both the display and stored video data, to cause the stored video to be represented upon a screen of display 200.

The use and manner of operation of a Humphrey field tester is well known in the art, and a description of such is therefore omitted in the interest of brevity. While a Humphrey field tester by Zeiss is shown, it should be understood that the disclosure can be carried out with other types or styles of automated VF testers, including those made by Zeiss, Oculus, Kowa, Synemed or Octopus, and can further be carried out using a manual system, such as a Goldmann tester, as detailed elsewhere herein.

In the embodiment of FIGS. 1 and 2, video display 200 is attached near the center or apex of perimeter 110 by any known means, including for example adhesive, resilient band, or hook and loop fastener. A removable fastener is advantageous in order to enable removal of display 200 when testing adults or other subjects, which are not in need of display 200, whereupon the default target can be used. For example, display 200 can be positioned directly over, around or adjacent to a preexisting fixation target light during testing. Display 200 can be of any size that is large enough to be viewed by the patient, and which does not obscure excessive portions of the perimeter that are to be tested.

Display 200 can be completely self-contained, wherein a video clip is manually started and stopped during testing. Alternatively, display 200 can be integrated into the computer control system 122 of tester 120, either by a plug-in cable or by a wireless method, such as a near field or BLUETOOTH network. In an embodiment, a portion of display 124 of tester 120 duplicates the video shown by display 200. In this manner, the examiner can verify that the correct video is being presented, and coordinate with the patient if the patient refers to the video. Additionally, the video can be paused by system 122 when testing is paused, and can be otherwise coordinated with the test procedure.

Testers 120 are known which include hardware and software to determine when the patient is not looking directly at the fixation target. Such hardware and software can be used in a like fashion to determine if the patient is not looking at display 200, which is positioned at the same location as the standard fixation target. Accordingly, when the patient is not looking at display 200, system 122 can play a video clip on display 200, and a corresponding audio clip, wherein the character depicted in display 200 asks the test subject, advantageously in a playful or amusing way, to continue to look at the display.

In tests carried out by the inventor, an entertaining video was presented upon display 200 during testing of children, and a statistically significant improvement in test reliability indices was noted, indicating increased attention and consistency of responses to test stimuli, which leads to increased VF test reliability. More particularly, children between ages 5 and 8 who are glaucoma suspects or have low vision were randomized to complete Humphrey VF testing with the video/audio intervention of the disclosure (‘the intervention’, n=4) versus without carrying out the disclosure, using standard equipment and methods (‘usual care’, n=3), and for each eye, at two visits about 1 week apart. A system as shown and described in FIGS. 1 and 2 was used, which included a 1.5″ micro-display video screen displaying popular cartoon characters as the fixation target, and audio clips of a corresponding cartoon-like voice. The audio clips were controlled by the test operator to provide specific instructional feedback to the child during testing, as described further below.

Results were analyzed, and mean false positive responses during VF testing at both visits were statistically significantly greater by 10% on average in children randomized to usual care testing compared to those who received the intervention (95% CI: 1 to 18%; p=0.03) after adjusting for age group and eye. At the second visit, false negatives were slightly greater in usual care controls by 16% on average in the second eye that was tested at the session when compared to those who received the intervention (95% CI: −2 to 34%; p=0.07).

From the results, it can be concluded that since false positives affect the accuracy and reliability of VF results, and that reductions in false positives using a test procedure in accordance with the disclosure can translate to improved diagnosis and care of young children with regard to potential peripheral vision loss. It should be noted that the disclosure can be used with any individual who, for whatever reason, has difficulty focusing on the fixation target, maintaining interest or concentration with respect to identifying stimuli, or otherwise participating in the test procedure in a reliable or repeatable way.

In an embodiment, video clips of about 10 minutes each are selected, and a different clip can be used when testing each eye. Video clips can be selected to be of greatest interest to the age and personality of the test subject. To maintain interest, the main character can be changed periodically, for example, every one to two minutes. Cartoon clips can be selected, for example, which feature close-ups of a character positioned centrally for several seconds as the character waves, smiles, or slightly moves around, in order to keep children engaged and fixated centrally, which may not be the case where multiple characters are present or the figures are changing locations.

A professional voice impersonator can be used to record a set of audio directions and feedback in an appropriate and engaging voice, such as a cartoon-type voice, which can be prepared and presented for each of the various cartoon characters, while the video is played during VF testing. During the VF test, the operator can use a computer or tablet device that contains these audio clips, which are advantageously organized into the following 6 categories, based on content:

(1) positive reinforcement to encourage the patient to continue to exert efforts for reliable test results (e.g. “Good Work!”);

(2) repositioning instructions, to promote proper and comfortable posture and body positioning of the patient (e.g. “Sit up, look straight at me, and we can start having fun!”);

(3) encouragement to reduce fatigue and positive emotions with respect to completing the test (e.g. “Almost done, you're doing so well, we'll be finished in no time!”);

(4) fixation instructions to keep the patient focused on the fixation target to improve reliability and reproducibility of the test (e.g. “Mr. Bunny hopes you'll watch him closely the whole time!”);

(5) stability instructions to maintain a consistent patient orientation to promote reliability and reproducibility of the test (e.g. “Please try to sit very still and look straight at me, so that you can get a high score!”); and

(6) instructional messages to advise the patient of what will take place, and what is expected of the patient (e.g. “Press the button when you see the dot appear, and Mr. Bunny gets another carrot!”).

While the visual field test is ongoing, the operator selects the appropriate type of audio clip to play based on the child's performance and behavior, as related to the 6 possible categories. Additional or fewer categories can be used, as determined by the medical practitioner.

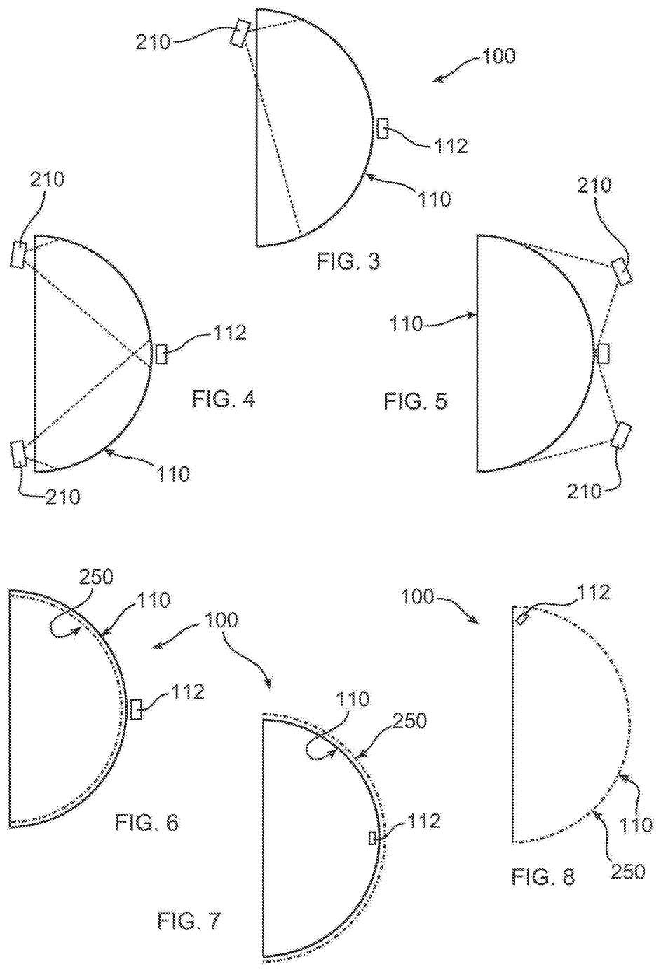

With reference to FIGS. 3-5, one or more projectors 210 can be provided to display the video clip and stimulus at appropriate locations upon perimeter 110. Perimeter 110 is a bowl shaped structure common to a variety of types and brands of VF testers, including the Humphrey type tester of FIG. 1, and other brands and styles of VF testers. Perimeter 110 can form a hemispherical bowl shape, as illustrated, or a smaller arc section of a bowl. Perimeter 110 does not necessarily form an arc of a circle, but can form some other arcuate shape, as best determined by the practitioner and the state of the art. Perimeter 110 can be any of the sizes that one may find in known VF perimeters, or perimeters that will be hereinafter developed, and need only be appropriately sized for adequately testing the peripheral range of vision of a desired demographic range of patients.

FIG. 3 depicts a single projector 210 oriented to project onto perimeter 110 from the front, while FIG. 4 illustrates two projectors having an overlapping projection area. An electronic processor, such as within system 122, or a separate computing device 700, such as a laptop or desktop computer, can coordinate the two projectors, and can skew or otherwise alter the projection so that the images appear in the correct locations and proportions from the perspective of the patient.

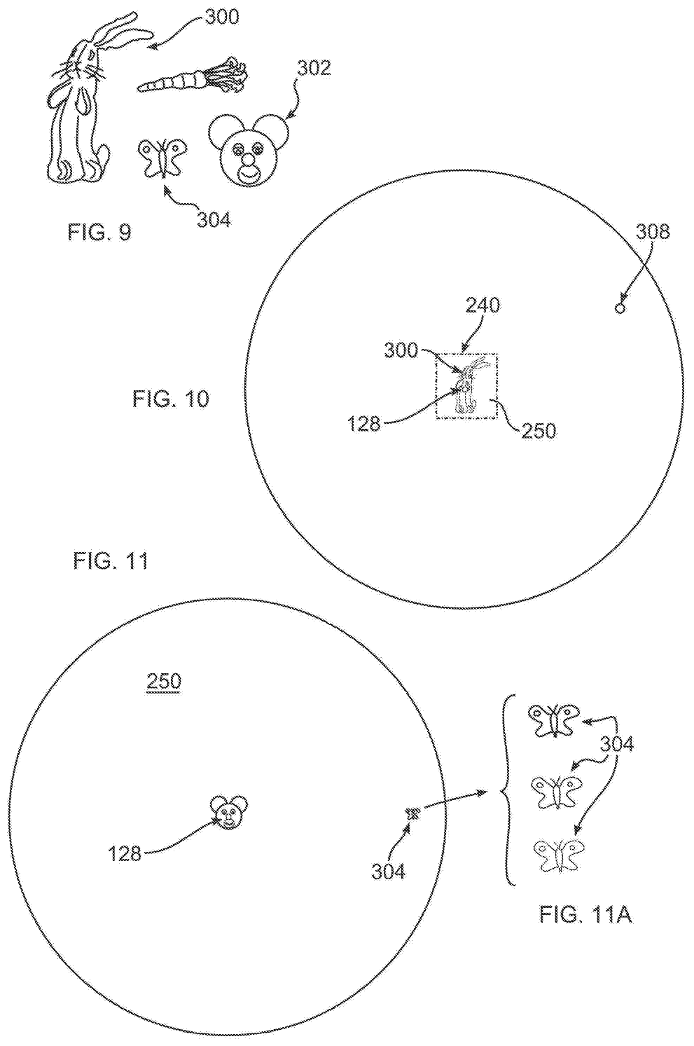

An eye orientation detector 112, such as a camera, is positioned so that the patient's eye can be observed by the operator on display 124 of the tester, or so that the eye can be electronically observed, in order to identify when the patient is not focused on the fixation target. An aperture 128 (shown in FIGS. 9-10) can be provided through a center of perimeter 110 to enable the patient's eye to be observed, although detector 112 can alternatively be placed within an interior of perimeter 110. When the eye is not focused on the fixation target, system 122 can cause the video clip to remind the child to look straight at the character, in an amusing way, and can automatically resume the test when the child has done so. While FIGS. 3-5 illustrate projection systems of the disclosure which can display all required elements, including the fixation target and stimulus target, any form of projection system known or herein disclosed can be combined with display 200. Some display elements are therefore shown using display 200, and others with a projection system or other display method. FIG. 1 depicts one such system. Display 200 can be comprised of more than one display type and technology.

In FIG. 5, perimeter 110 is fabricated using a rear projection screen material, and one or more projectors 210 are positioned behind perimeter 110. In the case of either front or rear projection configurations, display 200 can be eliminated, and the fixation target video clip is projected directly onto the center of perimeter 110, and stimulus objects are likewise projected onto perimeter 110.

In FIGS. 6-8, a flexible display 250 is used, and perimeter 110 can be lined with a flexible video display as depicted in FIG. 6; can support a rear mounted display as depicted in FIG. 7, for example by a transparent perimeter 110; or can form the display itself, as depicted in FIG. 8. In the latter embodiment, flexible display 250 can be made sufficiently rigid after being shaped to form perimeter 110. For example, the flexible display may be more flexible when heated, and when cool, retains a shape. When applied to an existing perimeter 110, flexible display 250 can be secured in place using any type of fastener, including clips, screws, hook and loop fastener, or adhesive. When flexible 250 is shaped to form perimeter 110, a frame or brace can be provided, or adhesive can be applied to the rear surface of the flexible display, to maintain the correct shape.

FIGS. 6-8 additionally depict various locations for detector 112, including an interior of perimeter 110 (FIG. 7), or an upper corner of perimeter 110, using for example a reflection of the patient's eye from perimeter 110.

Flexible display 250 can be fabricated using a rollable TFT-driven OLED display technology as demonstrated by SONY in partnership with RIKEN, or as developed by Arizona State University. Other examples of display technology that can be applied to perimeter 110, or which can be formed to create perimeter 110, are an organic thin film transistor (OTFT) technology color display by PLASTIC LOGIC, or a flexible AMOLED display by SAMSUNG. Alternatively, an electronic paper can be used, such as developed between HP and ASU using the Self-Aligned Imprint Lithography (SAIL) process. Other technologies may be hereinafter developed, which can alternatively be used to form flexible display 250. Further, it may be possible to manufacture flexible display 250 in the correct perimeter shape for system 100.

Flexible display 250 need only flex a single time, when it is applied to the interior or exterior curved surface of perimeter 110, or a similarly shaped mold, and is then held in place, for example using adhesive. As such, it may be possible to flex the display more than a recommended amount, as there will be no reverse flexing, or repeated flexing. If the selected display technology is limited in its ability to flex along more than one plane, multiple strips may be applied to perimeter 110. Likewise, if a single display for the selected technology cannot economically be made large enough to cover the desired peripheral diameter, multiple displays can be placed edge to edge to form a sufficiently large surface. It is advantageous for the display technology to be color enabled, although certain limited vision testing could be conducted with a grayscale display, if necessary. In an embodiment, strips which only bend along one plane or dimension can be used, provided that displayed stimulus objects are an equal or predetermined distance from the patient's eye.

Reported display curvatures are listed in Wikipedia (‘Flexible Display’), as reproduced in Table 1.

TABLE 1
List of displays by their reported curvature

| | | | Curved along its |
|---|---|---|---|
| | Diagonal | | wider/shorter |
| Model | (in) | Radius of curvature* | side? |
| Samsung Round | 5.7 | 400 millimeters (16 in) | shorter |
| LG G Flex | 6 | 700 millimeters (28 in) | wider |

*Lower is more sharply curved

It may be seen that the Samsung Round in particular, and also the LG G Flex, exhibit sufficient curvature to form a typical perimeter 110 diameter (e.g. 16 to 28 inches). If multiple flexible displays 250 are to be combined to form a radius of perimeter 110, the maximum radius of curvature of a single display can be larger than the total radius of the perimeter to be formed.

With reference to FIGS. 9-11, a use of cartoon characters is illustrated, together with flexible display 250. In FIG. 9, various cartoon elements are depicted, although it should be understood that any characters or images can be used, including characters or imagery that will appeal to older children or adults, who would benefit from the teachings of the disclosure. In FIGS. 10-11A, the example figures are displayed upon a projected display or flexible display 250, as viewed from the front (patient) side of a perimeter 110. A rabbit character 300 is shown at the center of the perimeter, corresponding to a desired fixation point. It is noted that rabbit 300 is illustrated to be quite small, to thereby cause the patient's gaze to be near the center of perimeter 110. As noted elsewhere, it can be advantageous for the character to form a compact focal point, such as the bear-like face 302 of FIG. 11. In both FIGS. 10 and 11, aperture 128 is provided to enable tracking of the direction of gaze of the tested eye, as described elsewhere herein, or an alternative eye position detector can be used, also as described elsewhere herein.

In FIG. 10, dashed line 240 indicates display 200 or flexible display 250 placed onto, inset within, or behind perimeter 110. An actual outline of the display may or may not be visible or perceptible to the patient or subject. An example stimulus 308 is shown, as normally projected or displayed by the VF test instrument.

In FIG. 11, the some or all of the perimeter 110 includes a flexible display 250, upon which is displayed both a central fixation target image 128 and a peripheral stimulus or other imagery in the periphery 304. It should be understood, however, that as one option, only a central fixation target image can be displayed as shown, with the stimulus target projected as is standard for the VF test instrument being used, but with another option to add peripheral images during a pause of the VF test to provide some interesting visual feedback and encouragement to the individual while they are taking a short break, or at the conclusion of the VF test to indicate good performance. However, in FIG. 11, a stimulus target 304, in this example in the shape of a compact butterfly, is displayed by projection or by flexible display 250, at various locations as would otherwise correspond to operation of a VF tester. A standard dot can alternatively be used. Alternatively, the stimulus can be displayed upon a colored background field. To ensure repeatability, it is important to electronically record or otherwise note which particular characters and/or background fields were used, so that test results can be properly compared for a given patient over time.

The stimulus target 304 can be displayed at any number of different brightness levels. While three brightness levels are illustrated in FIG. 11A, the number and intensity of brightness levels can be configured to correspond to known tests, or tests which may be hereinafter developed and which fully exploit the greater range of possible display elements enabled by the disclosure. More particularly, virtually anything can be displayed at any location upon a projected display or flexible display 250, with a wide variety of brightness levels, and with a high degree of resolution and precision.

Accordingly, the disclosure can enable the development of new tests, which have the potential to detect disorders earlier, and to track their progress with greater precision, thereby improving a quality of life for patients using the disclosure.

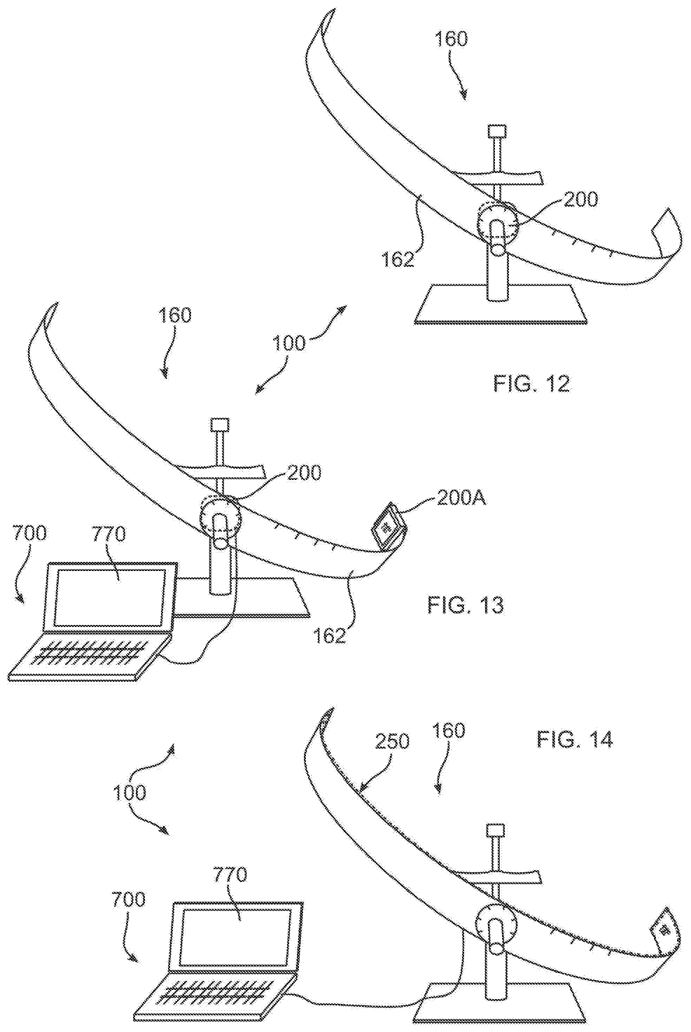

Referring now to FIGS. 12-14, a simple mechanical peripheral VF tester 160 is shown, which has been configured in accordance with the disclosure. The tester illustrated is intended to represent any form of mechanical tester, including an Arc perimeter tester. The type of simple example device illustrated in FIGS. 12-14 remains in use in many parts of the world today, and can be provided with greater accuracy and effectiveness when adapted or modified as described herein in accordance with the disclosure. Alternatively, such devices can be fabricated specifically as described herein.

In FIG. 12, display 200 has been positioned at a fixation target location at the center of the interior of a perimeter band 162 of tester 160. Display 200 is facing away when viewed in the drawing, towards a patient viewing the interior of the perimeter band 162. Display 200 can be caused to display a fixation video, as well as instructions, or any of the categories of audio content, all as described with respect to FIGS. 1-2, and the other embodiments herein. A stimulus target can be positioned at an appropriate central location for the particular type of tester. In the example embodiment, the typical stimulus object is a disk or light at the end of a wand. Display 200 can be attached to tester 160 by any known means, including releasable means such as an elastomeric band, clip, or hook and loop fasteners.

In FIG. 13, display 200 is associated with tester 160 as described with respect to FIG. 12, and is controlled by a laptop or other electronic computing device 700, using a wired or wireless connection, in a manner as described with respect to the processor of system 122 of tester 120. A copy of the video displayed upon display 200 can be simultaneously shown upon a display 770 of device 700. Device 700 can be used to select or control which video and audio clip is played, as described elsewhere herein.

In the embodiment of FIG. 12 or FIG. 13, a second display 200A can be clipped to perimeter 162, and can display a stimulus object of any form, and at various brightness levels as required by the selected test. Second display 200A can be moved by the practitioner to successive or different locations along perimeter band 162. If a computing device 700 is provided, second display 200A can be electronically connected by device 700 by a wire or wireless connection so that the stimulus image is selected by device 700.

Instructions can be provided upon display 770 of device 700 as to where to move second display 200A (or where to position a manual stimulus). In an embodiment, second display 200A includes a battery, controller, a motor, and wheels, gears, or other mechanical elements which enable display 200A to move along perimeter band 162 under control of device 700, in order to carry out a test of a patient, whereby the entire testing process is automated.

In FIG. 14, flexible display 250 is positioned along an interior of perimeter band 162, facing the patient, and the target and stimulus objects can be displayed as described with respect to FIGS. 6-8. Flexible display 250 can be comprised of a plurality of discreet displays, placed end to end along band 162, also as described with respect to FIGS. 6-8. A computing device 700 can control display 250 by wired or wireless connection, as described with respect to FIGS. 6-8 and elsewhere herein. As perimeter band 162 only requires bending along a single plane or dimension, a wider selection of display material is facilitated.

The embodiments of FIGS. 12-14 illustrate that the disclosure can be adapted to a wide variety of VF testers, and can produce a tester with the ability to be automated, and to more quickly and accurately test a patient's peripheral vision. Moreover, the adapted device can be used with individuals who have an insufficient attention span for testing, or who have another impairment which makes concentration during testing a challenge. Likewise, inexpensive and simple testing devices can be manufactured with displays of the disclosure, which can further be used with a computing device, to enable such devices to provide far better diagnostic capabilities than were previously available.

In subsequent testing of the device of FIG. 1, a device 100 that includes a microdisplay video screen 200 (1.5″ diameter) as the fixation target (instead of the standard LED light) was used to display edited video clips of popular cartoon characters, and a professional voice impersonator recorded audio clips of cartoon character voices presented by the test operator to provide instructional feedback based on children's performance as study participants during visual field (VF) testing. VF data was collected at two visits with children ages 5-8 years old, who were randomized to perform testing with device 100 (video/audio intervention) or usual care procedures for VF testing.

The study suggests that the intervention function as hypothesized and use of device 100 was very well received by the study participants. Mean false positive responses during VF testing at both visits were statistically significantly greater by 8% on average in children randomized to usual care testing compared to those who received the video/audio intervention (95% CI: 1-15%; p=0.03) after adjusting for age group, low vision, and eye. A previous publication suggested that VF results with false positive errors>20% should not be considered reliable [Vingrys & Demirel 1998]; none of the subjects with the video/audio intervention exceeded that rate. At the second visit, the video/audio intervention group subjects had 6% fewer false negatives for the second eye tested compared to the first eye, whereas usual care controls had 8% greater false negatives for the second eye (95% CI: −2%, 30%; p=0.07). This finding indicates that the usual care controls were more likely to be non-responsive to stimuli by the time they were completing the second eye, whereas the video/audio intervention group was more engaged and responsive on average.

The study team did not find any significant differences for these outcomes or acceptability of the intervention when comparing children with low vision compared to those with normal vision. Qualitative notes by the VF test operator indicated that the usual care controls typically had trouble sitting still during testing, required several pauses, tired quickly, thought it was boring, felt sleepy, did not like the test, and complained during testing. In contrast, the notes for the video/audio intervention group indicated that they were very cooperative, very interested, did not need breaks, were excited to play the ‘game’, said it was fun, liked doing the test, gave it a thumbs up at the end and actively wanted to come back to take the test again.

Example Computing System

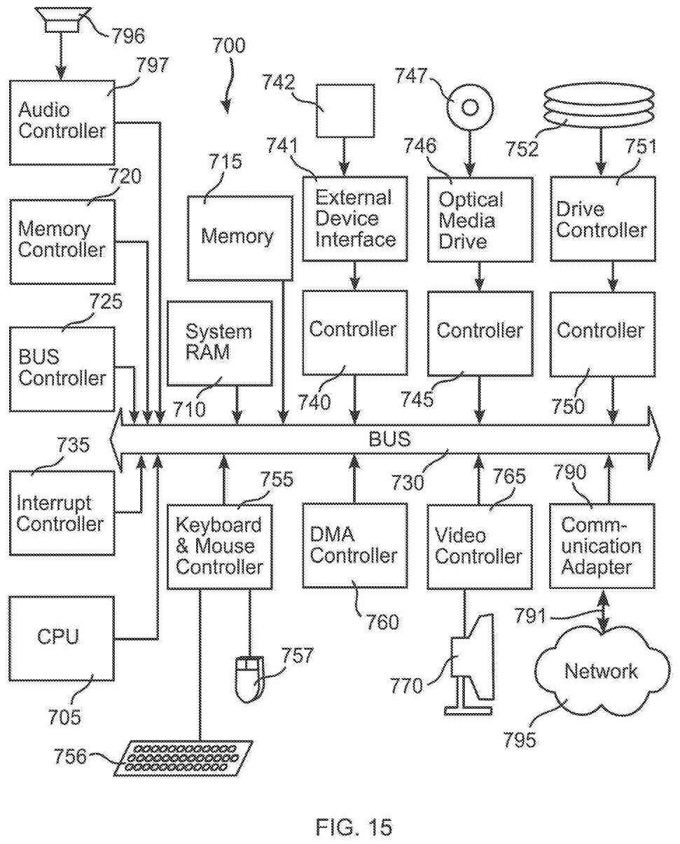

FIG. 15 illustrates the system architecture for a computer system 700, such as a process controller, or other processor on which or with which the disclosure may be implemented. The exemplary computer system of FIG. 15 is for descriptive purposes only. Although the description may refer to terms commonly used in describing particular computer systems, the description and concepts equally apply to other systems, including systems having architectures dissimilar to FIG. 15. Computer system 700 can control temperatures, motors, pumps, flow rates, power supplies, ultrasonic energy power generators, and valves, using actuators and transducers. One or more sensors, not shown, provide input to computer system 700, which executes software stored on non-volatile memory, the software configured to received inputs from sensors or from human interface devices, in calculations for controlling system 200.

Computer system 700 includes at least one central processing unit (CPU) 705, or server, which may be implemented with a conventional microprocessor, a random access memory (RAM) 710 for temporary storage of information, and a read only memory (ROM) 715 for permanent storage of information. A memory controller 720 is provided for controlling RAM 710.

A bus 730 interconnects the components of computer system 700. A bus controller 725 is provided for controlling bus 730. An interrupt controller 735 is used for receiving and processing various interrupt signals from the system components.

Mass storage may be provided by DVD ROM 747, or flash or rotating hard disk drive 752, for example. Data and software, including software 400 of the disclosure, may be exchanged with computer system 700 via removable media such as diskette, CD ROM, DVD, Blu Ray, or other optical media 747 connectable to an Optical Media Drive 746 and Controller 745. Alternatively, other media, including for example a media stick, for example a solid state USB drive, may be connected to an External Device Interface 741, and Controller 740. Additionally, another computing device can be connected to computer system 700 through External Device Interface 741, for example by a USB connector, BLUETOOTH connector, Infrared, or WiFi connector, although other modes of connection are known or may be hereinafter developed. A hard disk 752 is part of a fixed disk drive 751 which is connected to bus 730 by controller 750. It should be understood that other storage, peripheral, and computer processing means may be developed in the future, which may advantageously be used with the disclosure.

User input to computer system 700 may be provided by a number of devices. For example, a keyboard 756 and mouse 757 are connected to bus 730 by controller 755. An audio transducer 796, which may act as both a microphone and a speaker, is connected to bus 730 by audio controller 797, as illustrated. It will be obvious to those reasonably skilled in the art that other input devices, such as a pen and/or tablet, Personal Digital Assistant (PDA), mobile/cellular phone and other devices, may be connected to bus 730 and an appropriate controller and software, as required. DMA controller 760 is provided for performing direct memory access to RAM 710. A visual display is generated by video controller 765 which controls video display 770. Computer system 700 also includes a communications adapter 790 which allows the system to be interconnected to a local area network (LAN) or a wide area network (WAN), schematically illustrated by bus 791 and network 795.

Operation of computer system 700 is generally controlled and coordinated by operating system software, such as a Windows system, commercially available from Microsoft Corp., Redmond, Wash. The operating system controls allocation of system resources and performs tasks such as processing scheduling, memory management, networking, and I/O services, among other things. In particular, an operating system resident in system memory and running on CPU 705 coordinates the operation of the other elements of computer system 700. The present disclosure may be implemented with any number of commercially available operating systems.

One or more applications, such as an HTML page server, or a commercially available communication application, may execute under the control of the operating system, operable to convey information to a user.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

What is claimed is:

1. A device for perimetry visual field testing of a patient, comprising:
a perimeter having a target fixation location;
at least one video player positioned to display a video clip at the target fixation location;
an audio clip player positioned to play one or more audio clips to be heard by the patient during visual field testing;
an electronic controller associated with the video display to cause a particular video clip to play during testing.

2. The device of claim 1, wherein the audio clip is electronically synchronized to the video clip that is playing.

3. The device of claim 1, further including a plurality of audio clips containing messages corresponding to testing instructions.

4. The device of claim 1, wherein the display is an LCD, LED, or TFT type display.

5. The device of claim 1, wherein the particular video clip is selected from a plurality of cartoon or other imagery video clips.

6. The device of claim 1, wherein the particular video clip is selectable by a medical practitioner operating the device to test the patient.

7. The device of claim 1, wherein the display is projected onto at least one of the front and rear of the perimeter.

8. The device of claim 1, wherein the display is a flexible display which is curved to correspond to a shape of the perimeter.

9. The device of claim 1, wherein the perimeter and the video display are the same component.

10. The device of claim 1, wherein the perimeter forms part of a Humphrey type VF tester.

11. A method of visual field testing using a perimetry VF tester, comprising:
positioning a video display at a fixation location within a perimeter of the tester;
playing at least one video clips upon the video display during testing;
playing one or more audio clips corresponding to at least one of the video clip being displayed and a current stage of testing.

12. The method of claim 11, wherein the video and audio clip are a corresponding message of at least one of encouragement and testing instructions.

13. The method of claim 11, further including displaying a stimulus object using a video display.

14. The method of claim 13, wherein the video display at the fixation location extends to a peripheral location, and a video clip and the stimulus object are displayed using the video display.

15. The method of claim 11, further including using an electronic processor for controlling the test and for selecting the at least one video and audio clips for play during testing, the clips selected based upon events during testing.
